(19) United States
(12) Patent Application Publication
(10) Pub. No.: US 2016/0022661 A1
(43) Pub. Date: Jan. 28, 2016

(54) Dosage Form Comprising Crizotinib

(71) Applicant: RATIOPHARM GMBH, Ulm (DE)

(72) Inventors: Ralph STEFAN, Ebenweiler (DE); Sabine PROHL, Munich (DE)

(21) Appl. No.: 14/774,072

(22) PCT Filed: Mar. 13, 2014
(86) PCT No.: PCT/EP2014/054933

§ 371 Date: Sep. 9, 2015

Related U.S. Application Data
(60) Provisional application No. 61/780,015, filed on Mar. 13, 2013.

(30) Foreign Application Priority Data
Mar. 13, 2013 (EP) 13001257.8

(51) Int. Cl. A61K 31/4545; A61K 9/20; A61K 9/28

(52) U.S. Cl. CPC A61K 31/4545; A61K 9/2893; A61K 9/2095; A61K 9/2027; A61K 9/284

(57) ABSTRACT

The invention relates to a method of preparing a tablet, preferably a tablet for immediate release and having a high drug load, containing crizotinib in form of the free base and lubricant, both in specific amounts. The invention further relates to a tablet obtainable by said method.

### BACKGROUND OF THE INVENTION

The invention relates to a method of preparing a tablet, preferably a tablet for immediate release and having a high drug load, containing crizotinib in form of the free base. The invention further relates to a tablet obtainable by said method.

“Crizotinib” is reported to be the INN name of 3-[(1R)-1-(2,6-dichloro-3-fluorophenyl)ethoxy]-5-(1-piperidin-4-ylpyrazol-4-yl)pyridin-2-amine and is characterized by the following chemical formula (I):

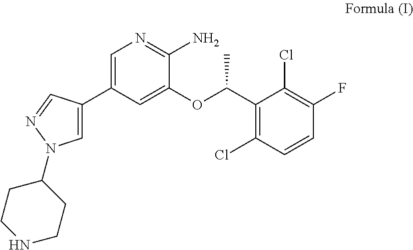 [SMILES: C[C@@H](Oc1cc(-c2cnn(C3CCNCC3)c2)cnc1N)c1c(Cl)ccc(F)c1Cl]

Crizotinib is reported to belong to the class of kinase inhibitors. Specifically, activity against the anaplastic lymphoma kinase (ALK) is reported, which can play different roles in oncogenesis.

EP 1 786 785 describes the synthesis of the compound crizotinib. WO 2007/066187 describes its use as c-Met/HGFR inhibitor. Further, WO 2007/066187 discloses some general information about pharmaceutical formulations, but lacks any detailed disclosure about any specific crizotinib dosage forms.

EP 1 963 302 describes an anhydrous crystalline crizotinib as well as crystalline crizotinib in form of a hydrate. Crystalline crizotinib exists in polymorphic forms, whereby Form 1 is described in detail.

WO 2013/181251 describes a variety of different crizotinib salts, inter alia crizotinib hydrochloride. It was described that such variations of the properties of different salts and solid state forms may provide a basis for improved formulation compared to the free base, for example, by facilitating better processing or handling characteristics, by improving the dissolution profile, or by improving stability (polymorph as well as chemical stability) and shelf life.

A dosage form for immediate release of crizotinib is commercially available and marketed under the trade name Xalkori®. Said dosage form is a hard gelatine capsule which contains a pharmaceutical formulation available as a powder, comprising 200 or 250 mg of the active pharmaceutical ingredient. The capsule which contains 250 mg crizotinib is reported to be a capsule with a capsule size of 0, which corresponds to a content of 0.7 ml. The high drug load capsule is quite voluminous due to the powdery formulation resulting from the API characteristics, such as low bulk and tapped density. Consequently, this dosage form is difficult to swallow, in particular, for older patients, which results in a poor patient compliance, especially of said patient group.

In view of the prior art, it was desirable to provide crizotinib in form of a tablet, in particular, in form of a tablet with a high drug load. Further, due to the described polymorphs, a wet preparation method should be avoided, since wet environment might facilitate the conversion of one polymorph into another, which might lead to an undesirable change of the dissolution profiles.

However, it turned out that the usual formulations for dry processes were not suitable for producing a tablet which contains a high amount of crizotinib or pharmaceutical salts thereof and has advantageous dissolution properties.

Hence, it was an object of the present invention to overcome the above problems.

Thus, it is an object of the present invention to provide a high drug load tablet, wherein any polymorph conversion should be avoided. Additionally, the tablet should show advantageous release properties, in particular, it should have at least the same or even a faster dissolution than Xalkori® capsules.

Furthermore, a tablet should be provided, wherein the tablet has a suitable size to be administered such that an increased patient compliance can be achieved.

### SUMMARY OF THE INVENTION

According to the present invention, the above objectives are achieved by a dry method for preparing a crizotinib tablet, wherein crizotinib is used in form of the free base, together with a specific and unusual high amount of lubricant in amounts described herein.

Thus, the subject of the present invention is a method for preparing a tablet comprising the steps of
i) providing crizotinib free base (a), lubricant (b), and optionally one or more pharmaceutical excipient(s);
ii) blending or dry-compacting the components from step i) with optionally one or more further pharmaceutical excipient(s);
iii) processing the mixture from step ii) into a tablet; and
iv) optionally film-coating the tablet,
   wherein the tablet comprises 20 to 70 wt % crizotinib free base and 5 to 25 wt % lubricant, based on the total weight of the tablet.

It was found that by the present method a tablet can be prepared in a size which is easy to swallow, so that an excellent patient compliance can be achieved. Further, the tablet of the present invention can be prepared without affecting important properties, such as the release of the active pharmaceutical agent.

The subject-matter of the present invention also relates to a tablet obtainable by the above method.

### DETAILED DESCRIPTION OF THE INVENTION

In the context of this invention the term “crizotinib” usually refers to 3-[(1R)-1-(2,6-dichloro-3-fluorophenyl)ethoxy]-5-(1-piperidin-4-ylpyrazol-4-yl)pyridin-2-amine in accordance with Formula (I) above. In addition, the term “crizotinib” as used in the present application can refer to crizotinib in the form of the free base as well as to its hydrates, solvates, polymorphs and mixtures thereof. It has been unexpectedly found that the use of the free base has advantages compared to the use of the salts, e.g. compared to the hydrochloric salt, e.g. with regard to dissolution properties.

In the present invention crizotinib is used in the form of the free base. Thus, within the present application the amounts or weight percent of crizotinib are based on the amount of crizotinib in form of the free base.

In a particularly, a preferred embodiment of the tablet of the invention comprises crizotinib as the sole pharmaceutically active agent. In another preferred embodiment the tablet of the invention can comprise crizotinib in combination with further pharmaceutically active agent(s).

A high drug load composition or high drug load dosage form can be any pharmaceutical composition or dosage form, wherein the amount of active pharmaceutical agent is 20 wt % or more. In a preferred embodiment of the invention crizotinib can be present in amounts of 20 to 75 wt %, more preferably 30 to 70 wt %, even more preferably 40 to 65 wt %, in particular, 45 to 63 wt %, especially preferred 46 to 59 wt %, based on the total weight of the present tablet.

Preferably, the tablet of the present invention comprises 50 to 1000 mg crizotinib, more preferably 100 to 750 mg crizotinib, still more preferably 150 to 500 mg crizotinib, particularly 200 to 300 mg crizotinib, most particularly 200 or 250 mg crizotinib, especially 250 mg crizotinib.

Crizotinib can be present in the present tablet in non-crystalline form or in crystalline form.

In a preferred embodiment crizotinib can be present in a non-crystalline form.

The term “non-crystalline” refers to any solid state being non-crystalline. Preferably, non-crystalline crizotinib means amorphous crizotinib, crizotinib in form of a solid dispersion or solid solution, in particular, amorphous crizotinib as compound (a) is preferred.

The term “amorphous” can be used in the context of this invention to designate the state of solid substances, in which the components (atoms, ions or molecules, i.e. in the case of amorphous crizotinib the crizotinib molecules) do not exhibit any periodic arrangement over a great range (=long range order). In amorphous substances the components are usually not arranged in a totally disordered fashion and completely randomly, but are rather distributed in such a way that a certain regularity and similarity to the crystalline state can be observed with regard to the distance from and orientation towards their closest neighbours (=short range order). Consequently, amorphous substances preferably possess a short range order but no long range order.

In contrast to anisotropic crystals, solid non-crystalline substances can be isotropic. Normally, they do not have a defined melting point, but instead pass over into the liquid state after slowly softening. They can be distinguished from crystalline substances experimentally by means of X-ray diffraction, since non-crystalline substances do not clearly reveal defined interferences, but rather in most cases only show few diffuse interferences with small diffraction angles.

In a particular preferred embodiment of the invention crizotinib is present in form of crystalline crizotinib.

The term “crystalline” can be used in the context of this invention to designate the state of solid substances in which the components (atoms, ions or molecules, i.e. in the case of crystalline crizotinib the crizotinib molecules) are arranged in an orderly repeating pattern, extending in all three spatial dimensions and thus exhibiting a periodic arrangement over a great range (=long range order).

The crizotinib (a) in the present tablet may consist of purely crystalline crizotinib. Alternatively, it may also contain small amounts of non-crystalline crizotinib components, provided that a defined melting point of crystalline crizotinib can be detected in a DSC. It is preferred that the crizotinib contained in the present tablet can be a mixture containing 85 to 99.999% by weight crystalline crizotinib and 0.001 to 15% by weight non-crystalline crizotinib, more preferably 90 to 99.99% by weight crystalline crizotinib and 0.01 to 10% non-crystalline crizotinib, particularly preferably 95 to 99.9% by weight crystalline crizotinib and 0.1 to 5% non-crystalline crizotinib.

The crizotinib comprised in the tablet of the present invention can have an average particle size (D50) of 0.5 to 150 μm, preferably 1.5 to 100 μm, more preferably 5 to 85 μm, particularly preferably 15 to 80 μm, especially 30 to 75 μm.

The average particle size can refer to the D50 value of the volume particle size distribution. The average particle can be determined by means of laser diffractometry. In particular, a Malvern Instruments Mastersizer 2000 can be used to determine the size (preferably wet measurement with ultrasound 60 sec., 2,000 rpm, preferably dispersed in sunflower oil, shadowing 10%, the evaluation being performed according to the Fraunhofer model).

The average particle size (D50), which is also denoted D50 value of the integral volume distribution, is defined in the context of this invention as the particle diameter at which 50 percent by volume of the particles have a smaller diameter than the diameter which corresponds to the D50 value. Likewise, 50 percent by volume of the particles have a larger diameter than the D50-value.

In a preferred embodiment of the invention the weight ratio of crizotinib (a) to lubricant (b) can be from 2:1 to 10:1, preferably from 2.3:1 to 8:1, more preferably from 2.5:1 to 7:1 and, particularly, from 3:1 to 6:1.

Lubricants (b) generally can be regarded as substances which are suitable to reduce friction, such as static friction, sliding friction and rolling friction. In particular, lubricants reduce the shearing forces which can occur on the borderline between tablet and mould, especially the sliding friction found during tablet pressing between the punch moving up and down in the die and the die wall on the one hand and between the edge of the tablet and the die wall on the other hand.

Lubricants can be present as a liquid or in a solid form, preferably in a solid form.

Lubricants used in the present invention preferably lead to an R-value of 0.90 to 0.99, preferably 0.92 to 0.985, more preferably 0.94 to 0.98.

The R-value is a measure for the effectiveness of the lubricant. Said R-value is determined on an instrumented eccentric press by comparing the force on the upper punch to the force on the lower punch (Strickland et al., J. Am. Pharm. Ass., Sci. Ed. 45, 51 (1956)).

The R-value is calculated from the following formula

R=force on the upper punch/force on the lower punch

Suitable lubricants are for example lauryl alcohol, myristyl alcohol, cetyl alcohol, stearyl alcohol, sodium stearyl fumarate, capric acid, lauric acid, myristic acid, palmitic acid, stearic acid, behenic acid and their derivatives, in particular esters such as aluminium stearate, calcium stearate, magnesium stearate, glyceryl monostearate, diglycol stearate, polyvinyl stearate, sorbitan stearate, polyethylene oxide stearate, glyceryl monopalmitate polyethylene oxide palmitate, polyvinyl myristate, sorbitan myristate, polyethyleneoxide myrisate, sorbitan laurate and glyceryl monobehenate, glyceryl dibehenate and glyceryl tribehenate.

Further, polyethylene glycol with a weight molecular weight of 1000 to 10000 g/mol can be used. The weight molecular weight can be determined by means of gel permeation chromatography. Additionally, defatted milk powder and talcum can be used as lubricant. Furthermore, inorganic lubricants on the basis of aluminosilicates, such as aluminumhydrosilicate (Gleitöl®) or Bolus alba are used.

In a preferred embodiment of the invention the lubricant can preferably be a hydrophobic lubricant. Examples of hydrophobic lubricants are salts of fatty acids, such as aluminium stearate, calcium stearate, magnesium stearate, zinc stearate, magnesium palmitate, calcium behenate, fatty acids with 11 to 22 carbon atoms, such as stearic acid, palmitic acids, hydrocarbons, such as paraffin, alcohols with 10 to 20 carbon atoms, such as stearyl alcohol, palmitoyl alcohol, myristyl alcohol, cetyl alcohol, talcum, olus alba, silicon talc, mono/di/tri glycerides of fatty acids, such as hydrated cotton seed oil (Sterotex®, Lubritab®) and hydrated castor bean oil (Cutina®HR), micro encapsulated vegetable or silicon oil, polytetrafluorethylen, phosphoric acid, tetraethylene glycol monomethyl ether alkyl sufonates, such as sodium doceylbenzene sulfonate and mixtures thereof. Magnesium stearate is preferred.

In an alternative preferred embodiment of the invention the lubricant can preferably be an amphiphilic lubricant. Examples of amphiphilic lubricants are glyceryl monostearate, a mixture of glyceryl monostearate and glyceryl distearate (Tegin 515®), glyceryl trimyristate (Dynasan 114®) glyceryl tripalmitate (Dynasan 116®), glycerin tristearate (Dynasan 118®), glyceryl monobehenate, glyceryl dibehenate and glyceryl tribehenate, glyceryl palmityl stearate (Precirol®), glyceryl fatty acid ester mixture (Boeson VP®), sorbitan stearate, sodium stearyl fumarate, saccharose monostearate, saccharose monopalmitate, sodium laurylsulfate, sodium laurylsulfate and mixtures thereof. Preferred are glyceryl dibehenate and sodium stearyl fumarate, in particular, glyceryl dibehenate.

In a preferred embodiment the lubricant (b) can comprise an organic residue containing 10 to 24 carbon atoms, preferably 14 to 22 carbon atoms, in particular, 18 to 22 carbon atoms.

It is particularly preferred that lubricant (b) is selected from glyceryl dibehenate, sodium stearyl fumarate, magnesium stearate and mixtures thereof.

In a dosage form of the present invention the lubricant (b) can preferably be one single lubricant.

In an alternative preferred embodiment of the invention lubricant (b) can be a mixture of different lubricants, in particular a mixture of two different lubricants. It is further preferred that the present dosage form comprises a mixture of at least one hydrophobic lubricant and a least one amphiphilic lubricant. A mixture of glyceryl dibehenate as amphiphilic lubricant and magnesium stearate as hydrophobic lubricant is particularly preferred.

The amount of amphiphilic lubricant is preferably from 0 to 100 wt %, more preferably from 40 to 90 wt %, in particular from 50 to 80 wt %, based on the total amount of lubricant.

The amount of hydrophobic lubricant is preferably from 0 to 100 wt %, more preferably from 20 to 90 wt %, in particular 30 to 50 wt %, based on the total amount of lubricant.

In an embodiment of the invention lubricant (b) can be present in amounts of 5 to 25 wt %, preferably 7 to 23 wt %, more preferably 9 to 20 wt %, in particular 11 to 17 wt %, based on the total weight of the tablet. In a preferred embodiment lubricant (b) can be present in amounts of 5 to 25 wt %, preferably 6 to 20 wt %, more preferably 7 to 18 wt %, in particular 8 to 15 wt %, based on the total weight of the tablet.

Generally, in the art lubricants are used in amounts of 1 to 3 wt %. Higher amounts of lubricants usually lead to undesired effects. However, it was found that the present tablet does not show any of the (negative) effects which are reported to be related to such high amounts of lubricant. For example, neither an influence on the (reduced) release, such as retardation, nor deterging effects, nor negative effects on the hardness of the resulting tablet can be observed, which usually occur when high amounts of lubricant are used.

In a preferred embodiment the present tablet can preferably comprise one or more pharmaceutical excipient(s). The pharmaceutical excipients are excipients with which the person skilled in the art is familiar, such as those which are described in the European Pharmacopoeia (Ph. Eur.) and/or in the US Pharmacopoeia (USP).

The present tablet can further comprise one or more pharmaceutical excipients, selected from glidants (c), fillers (d), disintegrants (e) and binders (f).

Glidants (c) can be used to improve the flowability. For example, talc can be used as glidant. More preferably, colloidal silicon dioxide (for example Aerosil®) is used. Preferably, the glidant can be present in an amount of up to 3 wt %, preferably in an amount of 0.05 to 2.5 wt %, more preferably 0.1 to 2.0 wt %, in particular 0.2 to 1.5 wt % based on the total weight of the present tablet. Preferably, the colloidal silicon dioxide has a specific surface area of 50 to 400 m²/g, measured by gas adsorption according to Ph. Eur., 6.0, Chapter 2.9.26.

Fillers (d) can be used to increase the bulk volume and weight to a limit at which a dosage form can be formed. Fillers may fulfil several requirements, such as being chemically inert, non-hygroscopic, biocompatible, easily processable and may possess good biopharmaceutical properties.

Preferred fillers are for example lactose, sucrose, glucose, mannitol, maltodextrin, dextrin, dextrose, hydrogenated vegetable oil and/or cellulose derivatives, such as microcrystalline cellulose and silicified microcrystalline cellulose, and mixtures thereof. More preferred are lactose, mannitol, microcrystalline cellulose and silicified microcrystalline cellulose, particularly lactose, microcrystalline cellulose and silicified microcrystalline cellulose, especially microcrystalline cellulose.

In an alternatively preferred embodiment of the invention the filler can be a high-density filler. A high-density filler is a filler which bulk density is from 0.9 to 1.5 g/cm³, preferably 1.1 to 1.45 g/cm³ and more preferably 1.2 to 1.4 g/cm³. Examples of high-density fillers are calcium phosphate, calcium hydrogen phosphate, calcium dihydrogen phosphate, calcium carbonate and magnesium carbonate, and mixtures thereof. Especially preferred are calcium phosphate and calcium hydrogen phosphate, in particular, calcium hydrogen phosphate.

The fillers can be present in the tablet of the present invention in an amount of 5 to 35 wt %, preferably 8 to 30 wt %, more preferably 12 to 27 wt % and still more preferably 15 to 25 wt % of the total weight of present tablet.

Disintegrants (e) are reported to be substances which accelerate the disintegration of a dosage form, especially of a tablet, after having been placed in water. Suitable disintegrants are, for example, organic disintegrants, such as carrageenan, croscarmellose sodium, sodium carboxymethyl starch and cross-linked polyvinylpyrrolidone (Kollidon Cl, crospovidone). Cross-linked polyvinyl-pyrrolidone (crospovidone) and/or croscarmellose sodium are particularly preferred.

Alternatively, inorganic alkaline disintegrants are used, preferably salts of alkali metals and alkaline metals. Preferred alkali and alkaline metals are sodium, potassium, magnesium and calcium. As anions, carbonate, hydrogen carbonate, phosphate, hydrogen phosphate and dihydrogen phosphate are preferred. The term “alkaline disintegrants” means disintegrants which produce a pH level of more than 7.0 when dissolved in water. Examples for inorganic alkaline disintegrants are sodium hydrogen carbonate, sodium hydrogen phosphate and calcium hydrogen carbonate.

Disintegrants can be present in an amount of 0 to 15 wt %, preferably 1 to 12 wt %, more preferably 2 to 10 wt % and still more preferably 3 to 8 wt %, based on the total weight of the tablet.

In a preferred embodiment of the invention the present tablet does not contain a disintegrant.

Binders (f) or adhesives are reported to be substances that ensure that granulates or tablets can be formed with the required mechanical strength. Binders can be, for example, starch, sucrose, gelatine, polyvinylpyrrolidone, cellulose derivatives, such as hydroxypropyl methylcellulose, or mixtures thereof.

Binders can be used in an amount of 0 to 15 wt %, preferably 2 to 12 wt %, more preferably 3 to 9 wt %, based on the total weight of the present tablet

In a preferred embodiment of the invention, the present tablet does not contain a binder.

In this regard it is generally noted that due to the nature of pharmaceutical excipients it cannot be excluded that a certain compound meets the requirements of more than one of the components (b) to (f).

However, in order to enable an unambiguous distinction, it is preferred in the present application that one and the same pharmaceutical compound can only function as one of the compounds (b) to (f). For example, if microcrystalline cellulose functions as filler (d), it cannot additionally function as disintegrant (e), even though microcrystalline cellulose also exhibits a certain disintegrating effect.

In a preferred embodiment of the invention the tablet can preferably comprise the following amounts of components (a) to (f).
(a) 20 to 75 wt %, preferably 30 to 70 wt %, more preferably 40 to 65 wt %, in particular 45 to 63 wt % crizotinib, especially 46 to 59 wt %,
(b) 5 to 25 wt %, preferably 7 to 23 wt %, more preferably 9 to 20 wt %, in particular 11 to 17 wt % lubricant,
(c) up to 3 wt %, preferably 0.05 to 2.5 wt %, more preferably 0.1 to 2.0 wt %, in particular 0.2 to 1.5 wt % glidant,
(d) 5 to 35 wt %, preferably 8 to 30 wt %, more preferably 12 to 27 wt %, in particular 15 to 25 wt % filler,
(e) 0 to 15 wt %, preferably 1 to 12 wt %, more preferably 2 to 10 wt %, in particular 3 to 8 wt % disintegrant, and
(f) 0 to 15 wt %, preferably 2 to 12 wt %, more preferably 3 to 9 wt % binder, and wherein the wt % are based on the total weight of the tablet.

In a preferred embodiment, the size of the present tablet can be 0.25 to 0.5 mL, preferably 0.28 to 0.4 mL.

In a preferred embodiment the tablet according to the invention provides an immediate release (“IR”) of crizotinib. This means that the release profile of the dosage forms of the invention according to USP app. I (basket, 900 ml, 0.1 n HCl, pH 1.2, 100 rpm, 37° C.) after 10 minutes usually indicates a content release of at least 70% or better 75%, preferably at least 85%, especially at least 90% and up to 100%.

In step (i) of the present method crizotinib (a), preferably crystalline crizotinib, and lubricant (b) and optionally one or more pharmaceutical excipient(s), preferably selected from glidant, filler, disintegrant and binder, can be present in the amounts described above. It is preferred that the lubricant is magnesium stearate or glyceryl dibehenate. Alternatively, a preferred lubricant (b) can be a mixture of at least two different lubricants as described above. It is preferred that at least one of the lubricants is an amphiphilic lubricant, in particular glyceryl dibehenate (Compritol® 888 ATO). Further, another lubricant can preferably be either a further amphiphilic lubricant, preferably sodium stearyl fumarate or a hydrophobic lubricant, preferably magnesium stearate.

It turned out that a mixture of at least two amphiphilic lubricants or a mixture of at least one amphiphilic and at least one hydrophobic lubricant can reduce the risk of a breakage of the punch and thus lower the scrap.

In a preferred embodiment of the invention step (i) can include preparing a pre-blend comprising crizotinib, lubricant and optionally a glidant. To prepare said pre-blend, crizotinib, lubricant and optionally a glidant are blended in a mixing device, preferably a tumble blender. All of said substances can preferably be sieved before the pre-blending. Crizotinib and glidant can preferably be blended before the lubricant is added and the pre-blend is formed. Lubricant can be preferably selected from glyceryl dibehenate (Compritol® 888 ATO), sodium stearyl fumarate, magnesium stearate and mixtures thereof. For preparing the pre-blend the whole amount of lubricant or part thereof can be used, preferably a part of lubricant is used, in which case the residual part of lubricant can preferably be blended in in one of the following steps. The glidant, used to prepare a pre-blend, can preferably be colloidal silicon dioxide (Aerosil® 200).

In a preferred embodiment, the pre-blend comprises 10 to 90 wt %, preferably 30 to 85 wt %, more preferably 50 to 80 wt %, in particular 55 to 75% wt % of the total amount of lubricant present in the tablet.

Preferably the pre-blend comprises,

40 to 95 wt %, more preferably 70 to 90 wt % crizotinib free base,

5 to 60 wt %, more preferably 10 to 30 wt % lubricant, and

0 to 30 wt %, more preferably 0 to 10 wt % glidant,

based on the total weight of the pre-blend.

It was unexpectedly found that the pre-blend can be advantageously used in the tabletting process. Hence, a further subject of the invention is the use of such a pre-blend comprising crizotinib free base and lubricant for producing an immediate release tablet, having desired properties, e.g. having a hardness of 50 to 325 N, measured according to Ph. Eur., 6.0, chapter 2.9.8, and/or a friability of less than 5%, particularly preferably less than 2 measured in accordance with Ph. Eur., 6.0, chapter 2.9.7. The explanations about preferred embodiments of the method of the present invention also apply for the use of the present invention.

In a preferred embodiment the components from step (ii), the components from step (i) and optionally one or more pharmaceutical excipients are blended. The blending of the above components can preferably be carried out in a mixer, preferably in a tumble blender.

In a preferred embodiment, the components from step (i) and/or (ii) can be sieved before being blended. In a preferred embodiment the sieve has a mesh size of 600 to 1400 μm, preferably of 800 to 1250 μm.

In another preferred embodiment in step (ii) a part of the one or more excipient(s), preferably sieved excipient(s), is added to the components from step (i) and subsequently blended. Subsequently, a further part of the one or more excipient(s), preferably sieved excipient(s), is added to the resulting blend and another blending is conducted. This procedure can be repeated, depending on the number of parts into which the one or more excipient(s) is/are divided.

Further, the mixture resulting from step (ii) preferably possesses a Hausner ratio in the range of 1.02 to 1.6, preferably of 1.08 to 1.4, more preferably of 1.10 to 1.20. The Hausner ratio is the ratio of tapped density to bulk density. Bulk density and tapped density can be determined according to USP 24, Test 616 “Bulk Density and Tapped Density”. A Hausner ratio within the above limits unexpectedly improved processability.

In an alternative embodiment of step ii) the mixture of step i) and optionally one or more further pharmaceutical excipient(s) is dry-granulated. Dry-granulation has the advantage of being more gentle for active agents and excipients. Furthermore, dry granulation, in particular, is an economical process.

“Granulating” is generally understood to mean the formation of relatively coarse or granular aggregate material by assembling and/or aggregating finer powder particles (agglomerate formation, or build-up granulation) and/or the formation of finer granules by breaking up coarser aggregates (disintegration, or break-down granulation).

Dry granulation is generally carried out by using pressure or temperature. Granulation is generally carried out in conventional granulating devices, such as roll granulators. Alternatively granulation can be carried out by bracketing via a tablet press.

Dry granulation is usually carried out as a continuous process.

In one embodiment of the process of the invention, in which dry granulation is contemplated, the mixture is compacted into a slug of material. The compacting conditions are preferably selected such that the compacted material has a density of 1.03 to 1.8 g/cm³, especially 1.05 to 1.7 g/cm³. The compacting is preferably carried out in a roll granulator. The rolling force per roll width in this case is preferably 2 to 50 kN/cm, more preferably 4 to 30 kN/cm, especially preferred 10 to 25 kN/cm. The gap width of the roll granulator is, for example, 0.8 to 5 mm, preferably 1 to 4 mm, more preferably 1.5 to 3 mm, especially 1.8 to 2.8 mm. After that, the compacted material is preferably granulated. The granulating can generally be performed by processes known in the state of the art. In step iii) the processing of the mixture from step ii) into a tablet can comprise compressing the mixture of step ii) into tablets.

Compressing the mixture from step ii) into tablets can be carried out by compressing said mixture on a press, for example on a rotary press, e.g. on a Fette® (Fette GmbH, Germany) or a Riva® Piccola (Riva, Argentina) or on an eccentric press, for example (Korsch EK0). In a preferred embodiment, step iii) comprises direct compression of the mixture from step ii). The direct compression of the mixture from step ii) avoids a granulation step and ensures a direct and easy procedure. The compression force can preferably range from 1 to 50 kN, preferably 3 to 40 kN.

The resulting tablets can have a hardness of 30 to 400 N, more preferred 50 to 325 N, still more preferred from 75 to 300 N, in particular from 85 to 275 N, wherein the hardness is measured according to Ph. Eur., 6.0, chapter 2.9.8.

In addition, the resulting tablets preferably can have a friability of less than 5%, particularly preferably less than 2%, especially less than 1%. The friability is determined in accordance with Ph. Eur., 6.0, chapter 2.9.7. The friability values generally refer to tablets without coating.

Finally, the tablets of the invention preferably can have a “content uniformity” of 90 to 110%, preferably 95 to 105%, especially 98 to 102% of the average content. The “content uniformity” is determined in accordance with Ph. Eur., 6.0, chapter 2.9.6. This means that each of twenty individual samples of the dosage form has a crizotinib content of between 90% and 110%, preferably 95% to 105%, even more preferably 98% to 102% of the average content of those twenty individual samples.

Hardness, friability and content uniformity are determined from an uncoated tablet.

In step iv) the tablet from step iii) can optionally be film-coated.

The tablet from step iii) can preferably be a tablet which can be swallowed unchewed (non-film-coated or preferably film-coated).

In a preferred embodiment, the tablet of the present invention can be film-coated. For this purpose, in step iv) methods known in the art for film-coating a tablet may be employed. Generally, film-coatings can be prepared by using cellulose derivatives, poly(meth)acrylate, polyvinyl pyrrolidone, polyvinyl acetate phthalate, and/or shellac or natural rubbers such as carrageenan.

In a preferred embodiment of the present invention the film-coating can be a film-coating, essentially without affecting the release of the active agent.

Preferred examples of film-coatings which do not affect the release of the active ingredient can be those including poly(meth)acrylate, methylcellulose (MC), hydroxypropyl methylcellulose (HPMC), hydroxypropyl cellulose (HPC), hydroxyethyl cellulose (HEC), polyvinyl pyrrolidone (PVP) and mixtures thereof. These polymers can have a weight average molecular weight of 10,000 to 150,000 g/mol.

The film coating can preferably have a thickness of 2 μm to 100 μm, preferably from 20 μm to 60 μm. In case of a coating containing crizotinib, the thickness of the coating is usually 10 μm to 200 μm, preferably from 50 μm to 125 μm.

It was unexpectedly found that tablets, which have been prepared by the method of the present invention, are significantly better suitable for film-coating (e.g. when compared with tablets prepared with lower amounts of lubricants).

Another subject of the present invention is a tablet obtainable by the method of the present invention. It is noted that the explanations given above for preferred embodiments of the process/method of the present invention (e.g. amount and type of crizotinib, lubricant and excipients) also apply for the tablet of the present invention.

The tablet of the invention are usually characterised by a release and absorption that lead to advantageous figures for the AUC (“area under curve”, the area under the curve of the plasma level 0 to 48 hours after peroral administration), advantageous figures for the Cₘₐₓ (maximum plasma level) and advantageous figures for the Tₘₐₓ (time when the maximum plasma level is reached after peroral administration).

In a preferred embodiment, the single-dose oral administration of the tablet of the present invention to a human patient can lead to a plasma level profile characterised by a Tₘₐₓ regarding the active agent crizotinib of about 4 to 6 hours.

In a preferred embodiment, the single-dose peroral administration of the present tablets comprising 250 mg crizotinib to a human patient can lead to a plasma level profile characterised by a Cₘₐₓ regarding the active agent crizotinib of about 40 to 150 ng/ml, preferably 75 to 145 ng/ml, in particular 120 to 135 ng/ml.

In a preferred embodiment, the single-dose peroral administration of the tablet of the present invention to a human patient can lead to a plasma level profile characterised by an AUC regarding the active agent crizotinib of about 800 to 3,500 ng×h/ml, preferably 2,000 to 3,000 ng×h/ml.

The above-mentioned plasma level figures are preferably average values, obtainable by examining blood samples from a group of 10 candidates (with an average body weight of 70 kg), the corresponding blood samples being taken 0, 1, 3, 4, 6, 8, 24 and 48 hours after the peroral single dose administration of the formulation of the invention. The plasma level values can be preferably achieved independently of the patient's food intake. The plasma level figures can preferably be determined by means of appropriate HPLC-MSMS methods. The AUC as described herein is the infinite AUC after a single dose and can be calculated by, for example, using a computer program such as the Microsoft Excel program.

Alternatively, the tablet of the present invention can preferably be administered twice daily. Steady state can preferably be reached within 15 days after multiple 250 mg BID in patients with cancers, with a mean AUCτ of 3.-6 to 4.0 ng·hr/mL and a Cₘₐₓ of 390 to 420 ng/mL on day 15 of cycle 1. No significant changes in C_{trough}, steady state following 250 mg BID were observed up to four treatment cycles with median in C_{trough}, steady state ranging from 230 to 330 ng/mL over day 15 to 112.

In a preferred embodiment, the tablet of the invention can be used to treat non-small cell lung carcinoma.

In an alternative preferred embodiment the tablet of the invention can be used to treat anaplastic thyroid cancer, tumors with ROS-1 mutations, i.e. wherein the cancer is mediated by at least one genetically altered ROS.

In a further alternative preferred embodiment the tablet of the invention can be used to inhibit ALK kinase activity.

The subject-matter of the invention is thus a tablet, containing 50 to 500 mg, preferably 200 to 250 mg crizotinib, preferably crystalline crizotinib, the dosage form having a content uniformity of 95 to 105%, and wherein the single dose administration regarding the active agent crizotinib leads to a Tₘₐₓ of 4 to 6 hours, a Cₘₐₓ of 40 to 150 ng/ml, more preferably 75 to 145 ng/ml, in particular 100 to 135 ng/ml and an AUC of about 800 to 3,500 ng×h/ml, preferably 2,000 to 3,000 ng×h/ml. Preferably, the dosage form is a tablet having a hardness of 50 to 250 N and a friability of less than 5%. In the tablet of the invention, crizotinib preferably is present in the form of the pharmaceutical composition of the invention.

The invention will now be explained with reference to the following examples.

### EXAMPLES

Preferred method for determination of the dissolution

| | |
|---|---|
| Method | USP, Typ I |
| Dosage | 250 mg crizotinib |
| Sample | tablets |
| Dissolution medium | 900 ml 0.1 n HCl, pH 1.2 |
| Temperature | 37° C. ± 0.5° C. |
| Agitation | basket 100 rpm |
| Sampling | on-line measurement |
| Total time | 90 minutes |
| Filter | 1 μm glass fibre membrane; |
| | 10 μm HDPE filter disks |
| Path length | 0.1 cm |
| Wave length | 220 nm |

### Example 1

Crizotinib was sieved (mesh size 800 μm) together with colloidal silicon dioxide and mixed together for 15 min in a tumble blender. The mixture was sieved (mesh size 800 μm) and glyceryl dibehenate and 50% of sodium stearyl fumarate were added through a sieve (mesh size 800 μm) and mixed together for 15 min in a tumble blender. This second blend was sieved (mesh size 800 μm) and microcrystalline cellulose and cross-linked polyvinylpyrrolidone (crospovidone, Kollidon CL) was added through a sieve (mesh size 800 μm) and mixed together for 15 min in a tumble blender. The residual amount of sodium stearyl fumarate was added through a sieve (mesh size 500 μm) and mixed for further 3 min in a tumble blender. The final blend was compressed on an eccentric press (Korsch EK0) to 15×7.5 mm oblong tablet with a hardness of approx. 60 to 100 N, wherein the tablets each contain

| | |
|---|---|
| Crizotinib free base | 250 mg (59.24%) |
| Glyceryl dibehenate | 40 mg (9.48%) |
| Sodium stearyl fumarate | 20 mg (4.74%) |
| Microcrystalline cellulose | 95 mg (22.51%) |
| Cross-linked polyvinylpyrrolidone | 15 mg (3.55%) |
| Colloidal silicon dioxide | 2 mg (0.47%) |

The tablets were coated with 15 mg Opadry II white by using the following equipment and parameters:

Lödige LHC 25

Inlet air: 40-50° C.

Outlet air: 30-45° C.

Product temperature: 35-45° C.

Nozzle: 1.2 mm

Spraying pressure: 1.5 bar

### Example 2

Crizotinib was sieved (mesh size 800 μm) together with colloidal silicon dioxide and mixed together for 15 min in a tumble blender. The mixture was sieved (mesh size 800 μm) and glyceryl dibehenate and 50% of magnesium stearate were added through a sieve (mesh size 800 μm) and mixed together for 15 min in a tumble blender. This second blend was sieved (mesh size 800 μm) and calcium hydrogen phosphate and crosslinked polyvinylpyrrolidone (crospovidone, Kollidon CL) were added through a sieve (mesh size 800 μm) and mixed together for 15 min in a tumble blender. The residual amount of magnesium stearate was added through a sieve (mesh size 500 μm) and mixed for further 3 min in a tumble blender. The final blend was compressed on an eccentric press (Korsch EK0) to 15×7.5 mm oblong tablets with a hardness of approx. 60-100 N, wherein the tablets each contain

| | |
|---|---|
| Crizotinib free base | 250 mg (59.24%) |
| Glyceryl dibehenate | 40 mg (9.48%) |
| Magnesium stearate | 20 mg (4.74%) |
| Calcium hydrogen phosphate | 95 mg (22.51%) |
| Cross-linked polyvinylpyrrolidone | 15 mg (3.55%) |
| Colloidal silicon dioxide | 2 mg (0.47%) |

The tablets were coated with 15 mg Opadry II white by using the following equipment and parameters:

Lödige LHC 25

Inlet air: 40-50° C.

Outlet air: 30-45° C.

Product temperature: 35-45° C.

Nozzle: 1.2 mm

Spraying pressure: 1.5 bar

### Example 3

| | |
|---|---|
| Crizotinib free base | 250 mg (58.69%) |
| Microcrystalline cellulose | 95 mg (22.30%) |
| Cross-linked polyvinylpyrrolidone | 15 mg (3.52%) |
| Colloidal silicon dioxide | 2 mg (0.47%) |
| Magnesium stearate | 64 mg (15.02%) |

Crizotinib and ⅔ of magnesium stearate were sieved (mesh size 800 μm) and mixed together for 15 min in a tumble blender. The mixture was sieved (mesh size 800 μm) once more. Microcrystalline cellulose (Avicel PH 102), cross-linked polyvinylpyrrolidone (Kollidon CL) and colloidal silicon dioxide (Aerosil 200) were added through a sieve (mesh size 800 μm) and the resulting mixture was blended together for 15 min in a tumble blender. The residual amount of magnesium stearate was added through a sieve (mesh size 500 μm) and the resulting mixture was blended together for 5 min in a tumble blender. The final blend was compressed on an eccentric press (Korsch EK0) to 15×7.5 mm oblong tablets with a hardness of approx. 100-150 N.

### Example 4

Crizotinib and ⅔ of magnesium stearate were sieved (mesh size 800 μm) and mixed together for 15 min in a tumble blender. The mixture was sieved (mesh size 800 μm) once more. Microcrystalline cellulose (Avicel PH 102), cross-linked polyvinylpyrrolidone (Kollidon CL) and colloidal silicon dioxide (Aerosil 200) were added through a sieve (mesh size 800 μm) and the resulting mixture was blended together for 15 min in a tumble blender. The residual amount of magnesium stearate was added through a sieve (mesh size 500 μm) and the resulting mixture was blended together for 5 min in a tumble blender. The final blend was compressed on an eccentric press (Korsch EK0) to 15×7.5 mm oblong tablets with a hardness of approx. 100-150 N, wherein the tablets each contain

| | |
|---|---|
| Crizotinib free base | 250 mg (62.11%) |
| Microcrystalline cellulose | 95 mg (23.60%) |
| Cross-linked polyvinylpyrrolidone | 15 mg (3.73%) |
| Colloidal silicon dioxide | 2 mg (0.50%) |
| Magnesium stearate | 40.5 mg (10.06%) |

### Example 5

Crizotinib and ⅔ of glyceryl dibehenate (Compritol ATO 888) were sieved (mesh size 800 μm) and mixed together for 15 min in a tumble blender. The mixture was sieved (mesh size 800 μm) once more. Microcrystalline cellulose (Avicel PH 102), cross-linked polyvinylpyrrolidone (Kollidon CL) and colloidal silicon dioxide (Aerosil 200) were added through a sieve (mesh size 800 μm) and the resulting mixture was blended together for 15 min in a tumble blender. The residual amount of magnesium stearate was added through a sieve (mesh size 500 μm) and the resulting mixture was blended together for 5 min in a tumble blender. The final blend was compressed on an eccentric press (Korsch EK0) to 15×7.5 mm oblong tablets with a hardness of approx. 100-150 N, wherein the tablets each contain

| | |
|---|---|
| Crizotinib free base | 250 mg (58.69%) |
| Microcrystalline cellulose | 95 mg (22.30%) |
| Cross-linked polyvinylpyrrolidone | 15 mg (3.52%) |
| Colloidal silicon dioxide | 2 mg (0.47%) |
| Glyceryl dibehenate | 64 mg (15.02%) |

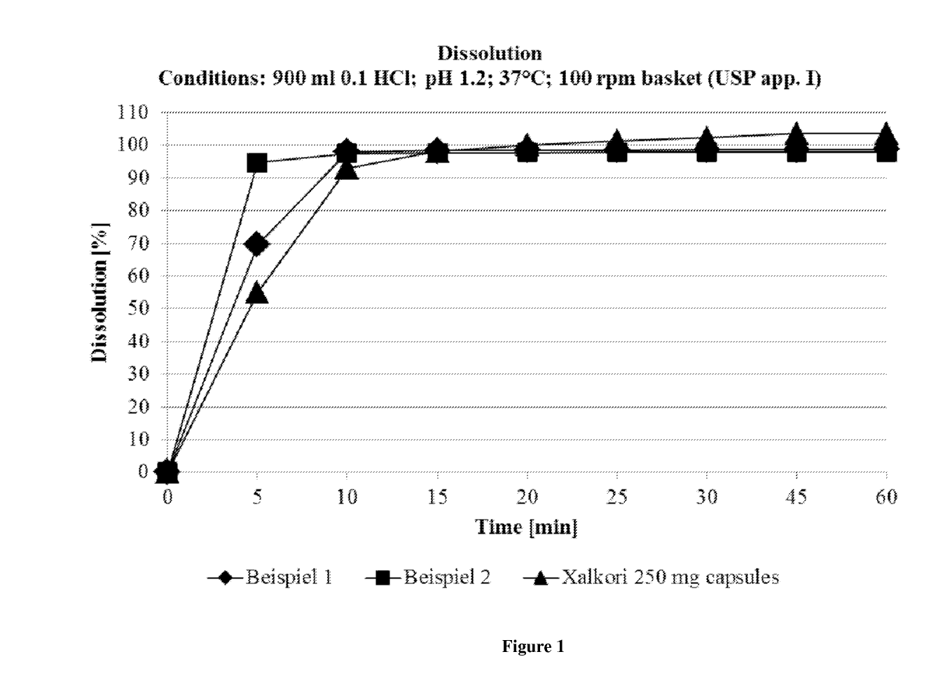

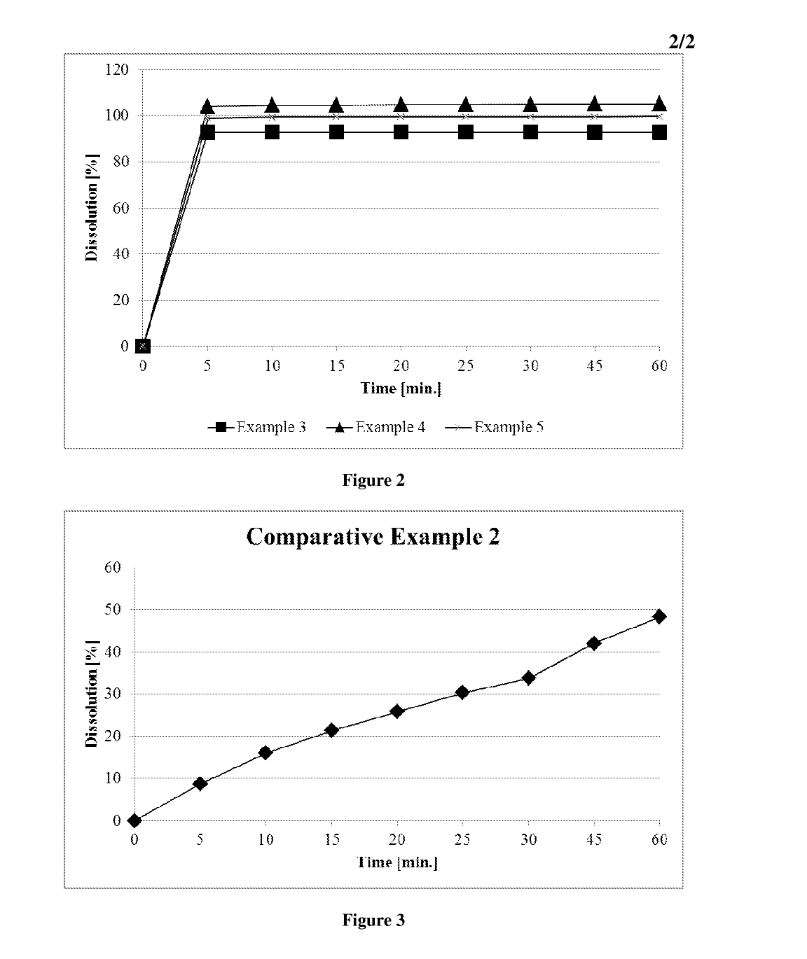

As can be seen from FIG. 1 the dissolution profiles of the tablets according to Examples 1 and 2 of the present invention comprising 250 mg crizotinib are at least as good as the dissolution profile of the Xalkori® capsules. In particular, after 5 min the present tablets show a dissolution of about 70% and about 95%, respectively, while the Xalkori® capsules show a dissolution of about 55%. Further, the size of the present tablets is a regular (small) tablet size. Compared to the prior art capsules, patient compliance could be increased with the tablets according to the present invention.

### Comparative Example 1

| | |
|---|---|
| Crizotinib free base | 250 mg (62.11%) |
| Microcrystalline cellulose | 120 mg (31.46%) |
| Cross-linked polyvinylpyrrolidone | 15 mg (3.73%) |
| Colloidal silicon dioxide | 2 mg (0.50%) |
| Magnesium stearate | 15.5 mg (3.84%) |

Crizotinib was sieved (mesh size 800 μm). Microcrystalline cellulose (Avicel PH 102), cross-linked polyvinylpyrrolidone (Kollidon CL) and colloidal silicon dioxide (Aerosil 200) were added through a sieve (mesh size 800 μm) and the resulting mixture was blended together for 15 min in a tumble blender. Magnesium stearate was added through a sieve (mesh size 500 μm) and the resulting mixture was blended together for 5 min in a tumble blender. It was tried to compress the final blend on an eccentric press (Korsch EK0) to obtain 15×7.5 mm oblong tablets. However, parts of the tableting mass remained stuck to the punches such that an appropriate tableting process could not be performed.

### Comparative Example 2

Crizotinib and ⅔ of magnesium stearate were sieved (mesh size 800 μm) and mixed together for 15 min in a tumble blender. The mixture was sieved (mesh size 800 μm) once more. Microcrystalline cellulose (Avicel® PH 102), cross-linked polyvinylpyrrolidone (Kollidon CL) and colloidal silicon dioxide (Aerosil 200) were added through a sieve (mesh size 800 μm) and the resulting mixture was blended together for 15 min in a tumble blender. The residual amount of magnesium stearate was added through a sieve (mesh size 500 μm) and the resulting mixture was blended together for 5 min in a tumble blender. The final blend was compressed on an eccentric press (Korsch EK0) to 15×7.5 mm oblong tablets with a hardness of approx. 100-150 N, wherein the tablets each contain

| | |
|---|---|
| Crizotinib HCl | 250 mg (62.11%) |
| Microcrystalline cellulose | 90 mg (22.34%) |
| Cross-linked polyvinylpyrrolidone | 15 mg (3.73%) |
| Colloidal silicon dioxide | 2 mg (0.50%) |
| Magnesium stearate | 45.5 mg (11.30%) |

The dissolution of the obtained tablets was rather poor (16% in 10 minutes) and was significantly slower than that of the Xalkori® capsules.

1. A method for preparing a tablet comprising the steps of
i) providing crizotinib free base (a) and lubricant (b);
ii) blending or dry-compacting the components from step i); and
iii) processing the mixture from step ii) into a tablet;
wherein the tablet comprises 20 to 70 wt % crizotinib free base and 5 to 25 wt % lubricant, based on the total weight of the tablet.

2. The method according to claim 1, wherein the crizotinib free base (a) is present from 40 to 65 wt %, based on the total weight of the tablet.

3. The method according to claim 1, wherein the crizotinib has an average particle size (D50) of 0.5 to 150 μm.

4. The method according to claim 1, wherein the weight ratio of crizotinib (a) to lubricant (b) is from 2:1 to 10:1.

5. The method according to claim 1, wherein the lubricant produces an R-value from 0.90 to 0.99.

6. The method according to claim 1, wherein the lubricant comprises an organic residue containing 10 to 24 carbon atoms.

7. The method according to claim 1, wherein lubricant is amphiphilic.

8. The method according to claim 1, wherein the tablet further comprises one or more pharmaceutical excipients.

9. The method according to claim 1, wherein the tablet comprises
a) 20 to 75 wt % crizotinib free base,
b) 5 to 25 wt % lubricant,
c) 0.1 to 3 wt % glidant,
d) 5 to 35 wt % filler,
e) 0 to 15 wt % disintegrant, and
f) 0 to 15 wt % binder,
and wherein the wt % are based on the total weight of the dosage form.

10. The method according to claim 1, wherein the tablet provides immediate release of crizotinib.

11. The method according to claim 1, wherein step i) includes preparing a pre-blend comprising crizotinib and lubricant.

12. The method according to claim 1, wherein step iii) comprises compression of the mixture from step ii).

13. A tablet obtained by the method according to claim 1.

14. Use of a pre-blend comprising crizotinib free base and lubricant for producing an immediate release tablet having a hardness of 50 to 325 N, measured according to Ph. Eur., 6.0, chapter 2.9.8, and/or a friability of less than 5%, measured in accordance with Ph. Eur., 6.0, chapter 2.9.7.

15. The method according to claim 1, further comprising film coating the tablet.

16. The method according to claim 8, wherein the one or more pharmaceutical excipients are selected from the group consisting of glidants (c), fillers (d), disintegrants (e), binders (f) and combinations of (c)-(f).

17. The method of claim 11, wherein the pre-blend further comprises a glidant.

18. The intermediate release tablet of claim 14, wherein the friability is less than 2%.
